(19) United States
(12) Patent Application Publication
(10) Pub. No.: US 2014/0364877 A1
(43) Pub. Date: Dec. 11, 2014

(54) HERNIA PATCH ANCHOR

(71) Applicant: Roderick B. Brown, Glenwood, MN (US)

(72) Inventor: Roderick B. Brown, Glenwood, MN (US)

(21) Appl. No.: 14/289,037

(22) Filed: May 28, 2014

Related U.S. Application Data
(60) Provisional application No. 61/832,292, filed on Jun. 7, 2013.

(51) Int. Cl. A61B 17/064; A61F 2/00

(52) U.S. Cl. CPC A61B 17/0644; A61F 2/0063; A61B 2017/0647
USPC 606/151

(57) ABSTRACT

An anchor for adhering a mesh patch to a defect in an abdominal wall in the course of a hernia repair procedure comprises a tubular metal shank having a radial flange at one end and a pair of deployable wing members hinged to the shank at its opposite end, the wings when undeployed cooperating with one another to form a pointed tip end to facilitate passage through the mesh patch, a layer of fascia and into muscle tissue. The anchor further includes a manual actuator for spreading the pair of wings apart to extend parallel to the radial flange.

### CROSS-REFERENCED TO RELATED APPLICATIONS

The present application claims priority to provisional application, Ser. No. 61/832,292, filed Jun. 7, 2013, and which is hereby incorporated herein by reference.

### STATEMENT REGARDING FEDERALLY SPONSORED RESEARCH OR DEVELOPMENT

Not applicable

### BACKGROUND OF THE INVENTION

I. Field of the Invention

This invention relates generally to laparoscopic hernia repair and more particularly to an improved anchor for securing surgical mesh in place in the treatment of ventral hernias.

II. Description of the Prior Art

A ventral hernia is a bulge through an opening in the muscle on the abdomen. If the hernia reduces in size when a person is lying flat or in response to manual pressure, it is reducible. If it cannot be reduced, it is said to be incarcerated and a portion of the intestine may be bulging through the hernia sac. A hernia is said to be strangulated if the intestine is trapped in the hernia pouch and the blood supply to the intestine is decreased.

A primary abdominal hernia occurs spontaneously in the abdomen. An incisional hernia bulges through a past incision site. This issue can be the result of scar tissue or weak muscles around the earlier surgical site.

The type of surgical treatment depends on the hernia size, location, and if it is a repeat hernia.

In an open surgical repair procedure, the surgeon makes an incision near the hernia site. The bulging tissue is gently pushed back into the abdomen. Sutures, mesh or a tissue flap is used to close the muscle. With complex or large hernias, small drains may be placed going from inside to the outside of the abdomen. The site is closed using sutures, staples or surgical glue.

In a laparoscopic hernia repair, the surgeon will make several small punctures or incisions in the abdomen. Ports or trocars (hollow tubes) are inserted into the openings. Surgical tools, including an endoscope, are placed into the ports. The abdomen is inflated with carbon dioxide gas to make it easier for the surgeon to view the hernia. A mesh patch is often sutured, stapled or clipped to the muscle around the hernia site. The hernia site can also be sown directly together.

Because it is somewhat difficult in a laparoscopic procedure to suture a mesh patch in place, a tool has been developed for placing coil screws or tacks through the mesh material and into only the innermost millimeters of the peritoneal cavity.

Prior art tacks used in laparoscopic hernia repair for securing a mesh patch in place have several drawbacks.
Sharp/rough surface—actually will tear surgical gloves.
The intra-peritoneal surface of the tack/screws promotes adhesion formation.
Small diameter of tacks/screws is ineffective with porous mesh materials—mesh call pull through.
Short length tacks/screws limit tissue purchase with thicker mesh materials.
Optimal mesh support often requires additional support i.e., trans-fascial sutures.
Tiny area of mesh/tissue contact afforded by tacks/screws limits their effectiveness of support.
Delivery systems for prior art tacks/screws make placement perpendicular to mesh/abdominal wall needed for ventral hernia repair difficult or impossible.

Therefore, a need exists for an improved anchor device for securing a hernia patch in place that is easy to deploy, that does not result in adhesion formation and that can be readily removed should it become necessary to remove the mesh should infection occur.

### SUMMARY OF THE INVENTION

The improved anchor of the present invention comprises a short, tubular shank segment having first and second ends and with a radially extending flange surrounding the first end and with at least two wing members hinged to the shank's second end. The wing members form a generally cylindrical spike when closed against one another, but which can be spread apart to lie generally parallel to the circular flange of the flange upon deployment of a wing spreading actuator. In use, the anchor, with its wings closed against one another, is pushed through the mesh and through the fascia into the underlying muscle until the flange engages the mesh. Next, the wing spreading actuator is displaced relative to the lumen of the tubular shank which spreads each of the wings approximately 90° so as to lay flat and generally parallel to the flange to thereby resist unwanted expulsion.

The wing spreading actuator preferably comprises one or more strands of Nitinol wire which have been heat-treated to impart an arcuate bend which are rendered rectilinear when confined by the tubular shank but which exerts a bending force on the wing members sufficient to deploy the wings to their spread state when manually advanced in a distal direction from the confines of the tubular shank.

### DESCRIPTION OF THE DRAWINGS

The foregoing features, objects and advantages of the invention will become apparent to persons skilled in the art from the following detailed description of a preferred embodiment, especially when considered in conjunction with the accompanying drawings in which:

FIG. 1 is a greatly enlarged side elevation of the mesh anchor of the present invention;

FIG. 2 is a greatly enlarged bottom view thereof;

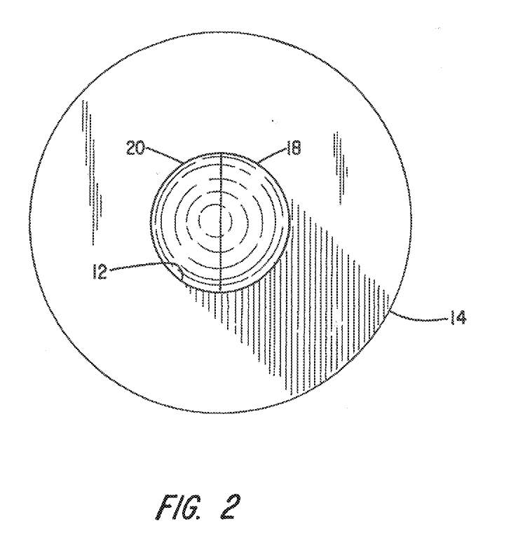

FIG. 3 is a side elevation of the anchor of FIG. 1 with a wing deployment actuator added; and

FIG. 4 shows the anchor when fully deployed.

### DESCRIPTION OF THE PREFERRED EMBODIMENT

This description of the preferred embodiments is intended to be read in connection with the accompanying drawings, which are to be considered part of the entire written description of this invention. In the description, relative terms such as “lower”, “upper”, “horizontal”, “vertical”, “above”, “below”, “up”, “down”, “top” and “bottom” as well as derivatives thereof (e.g., “horizontally”, “downwardly”, “upwardly”, etc.) should be construed to refer to the orientation as then described or as shown in the drawings under discussion. These relative terms are for convenience of description and do not require that the apparatus be constructed or operated in a particular orientation. Terms such as “connected”, “connecting”, “attached”, “attaching”, “join” and “joining” are used interchangeably and refer to one structure or surface being secured to another structure or surface or integrally fabricated in one piece, unless expressively described otherwise.

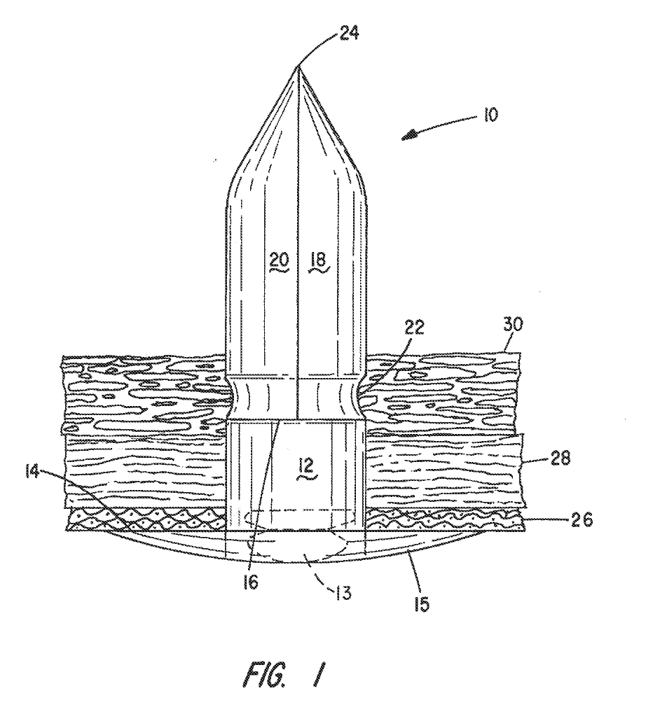

Referring to FIG. 1, the mesh anchor of the present invention for use in adhering a mesh reinforcing patch with respect to a hernia defect is indicated generally by numeral 10 and is seen to comprise a tubular shank member 12 to which a radially extending annular flange 14 is integrally formed or affixed, the flange being generally circular and with a central opening as seen in FIG. 7. Hinged to the upper end 16 of the shank 12 are foldable wing members 18 and 20. While only two such wings are shown, it is possible to form the anchor with three or even four such wings.

The lower end portion of the shank 12 includes an inwardly extending tapered ring 13.

The anchor 10 may be made from a suitable metal such as titanium or may be of a suitable medical grade plastic, such as Peek. A living hinge, as at 22, is formed by reducing the thickness of the material to create a circumferential, preferential fold line. Alternatively, a separate hinge member can be used. The wings 18 and 20 are shaped so that when erect, as shown in FIG. 1, they come together to form a conical tip portion ending at a point 24. Being so shaped, it allows the anchor to more readily penetrate through a mesh layer 26 and through fascia tissue 28 into a muscle 30.

With continued reference to FIG. 1, the flange 14 preferably has a smooth gently rounded contour, as at 15, with no sharp edges that might erode tissue structures that come in contact with it.

Without limitation, and strictly for the purpose of example, the flange may have a diameter in the range of from 4 to 10 mms while the tubular shank 12 may have an outer diameter in the range of from 1 to 4 mms. The wings 18 and 20 are preferably in a range of from 5 to 10 mms in length.

Once the anchor device of FIG. 1 has been inserted, means are provided for folding the wings 18 and 20 so as to extend generally parallel to the flange 14 while residing in muscle.

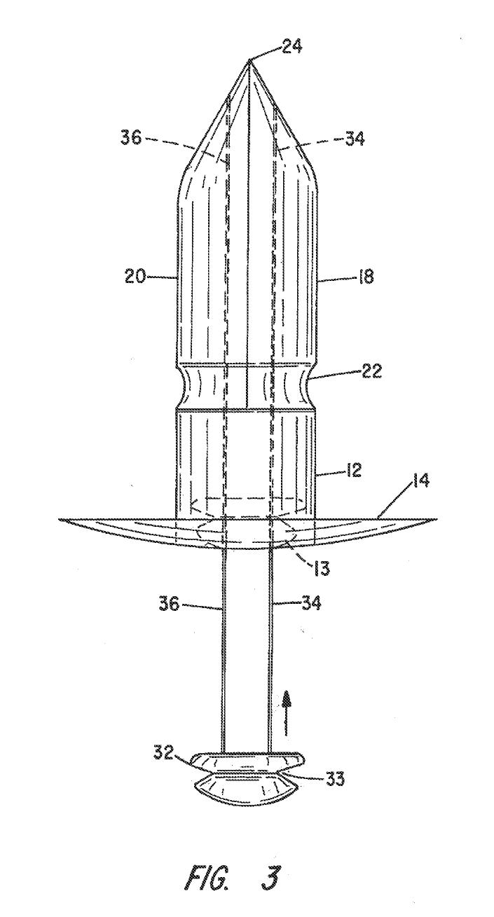

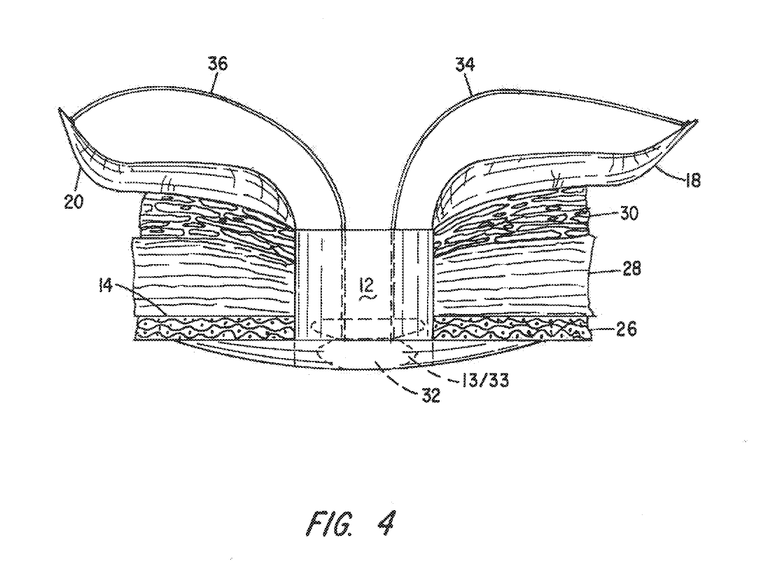

In FIG. 3, there is shown a built-in actuator for deploying the wings to their folded position illustrated in the drawing of FIG. 4. The actuator may comprise a spherical base serving as a handle or grip 32 to which are affixed a pair of metal wire strands or rods 34, 36 that extend through the lumen of the shank 12 and are affixed to the wing members 18 and 20 proximate an upper end thereof when viewed as in FIG. 3. The rods or wire strands 34, 36 preferably comprise Nitinol® alloy that exhibits a strong superelastic memory property that in a heat treat process is initially formed to exhibit arcuate end portions when unconstrained.

As those skilled in the art appreciate, a superelastic alloy, when mechanically loaded, deforms reversibly to high strains by the creation of a stress-induced phase. When the load is removed, the stress-induced phase becomes unstable and the material regains its original shape and, in the case of superelastic alloys, no temperature change is need for the alloy to recover its initial shape.

The semi-spherical base or handle 32 includes an inwardly extending V-shaped groove or recess 33 that is adapted to cooperate with the tapered ring 13 on the shank 12 to function as a detent.

With reference to FIGS. 3 and 4, the deployment actuator comprising the Nitinol strands 34 and 36 is fitted through the central lumen of the shank 12. Initially, the stress provided by engagement with the lumen walls of the shank 12 maintains the strands 34, 36 rectilinear and, as they are advanced distally, the end portions of the actuator are free to bend to their preformed arcuate curvature. In doing so, the strands 34, 36 apply a force to the wing members 18 and 20 sufficient to spread them apart to the disposition illustrated in FIG. 4. This firmly captures the mesh patch material between the circular flange 14 and the fascia tissue layer.

The actuator element will remain in place with the semi-spherical base 32 residing partially within the lumen of the shank 12 and held by the detent as seen in FIG. 4. The rounded contour of the semi-circular base 32 conforms to the curvature of the flange when latched in place by the detent.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. A surgical tack for securing a mesh patch in place during the course of a ventral hernia repair procedure comprising:
(a) a tubular shank member with first and second ends;
(b) a flange projecting radially from the tubular shank proximate the first end; and
(c) a pair of wing members affixed to the tubular shank proximate the second end, the pair of wing members being deployable from a first state generally aligned with the tubular shank and a second state generally parallel to the flange.

2. The surgical tack of claim 1 wherein the pair of wing members are affixed to the tubular shank by a living hinge.

3. The surgical tack of claim 1 wherein each of the pair of wing members are generally semi-circular in cross-section and tapered proximate a distal end so as to cooperate with one another to form a pointed tip when in the first state.

4. The surgical tack of claim 2 wherein the pair of wing members are generally semi-circular in cross-section and cooperate with one another to form a pointed tip when in the first state.

5. The surgical tack of claim 3 and further including a means for deploying the wing members from the first state to the second state.

6. The surgical tack of claim 5 wherein the means for deploying the wing members from the first state to the second state comprises a displaceable element extending through the tubular shank and cooperating with the pair of wing members proximate the distal ends thereof.

7. The surgical tack of claim 6 wherein the displaceable element comprises first and second strands of a shape memory material extending from a base member.

8. The surgical tack of claim 7 wherein the shape memory material is a Nitinol alloy, said strands exhibiting superelastic properties such that displacement through the tubular shank results in the strands resuming a pre-programmed shape and spreading the pair of wing members to the second state.

9. The surgical tack of claim 1 wherein the shank has a length sufficient to extend through a layer of surgical mesh patch material, a layer of fascia and into an abdominal muscle when used in a ventral hernia repair procedure.

10. The surgical tack of claim 3 wherein the wing members have a length between the living hinge and the distal ends thereof in a range of from 5 mm to 10 mm, the flange has a diameter in a range of from 4 mm to 10 mm and the shank has a diameter in a range of from 1 mm to 4 mm.

11. The surgical tack of claim 7 and further including a detent member operatively coupling the base member to the shank upon displacement of the displaceable element.
